(19) United States
(12) Patent Application Publication
(10) Pub. No.: US 2025/0325171 A1
(43) Pub. Date: Oct. 23, 2025

(54) FILTER CHANGING APPARATUS FOR AN ENDOSCOPIC CAMERA, CAMERA HEAD FOR AN ENDOSCOPE, AND RETROFIT KIT FOR RETROFITTING A CAMERA HEAD AND/OR AN ENDOSCOPE

(71) Applicant: KARL STORZ SE & Co. KG, Tuttlingen (DE)

(72) Inventor: Jonas DAVID, Tuttlingen (DE)

(21) Appl. No.: 19/182,710

(22) Filed: Apr. 18, 2025

(30) Foreign Application Priority Data
Apr. 18, 2024 (DE) 10 2024 110 880.0

(51) Int. Cl. A61B 1/00; A61B 1/04

(52) U.S. Cl. CPC A61B 1/00186; A61B 1/00131; A61B 1/042; A61B 2560/0462

(57) ABSTRACT

The invention relates to a filter changing apparatus for an endoscopic camera, wherein a first rotating plate bearing with two recesses for the passage of one guide element each is arranged between a first housing part and a first plate surface of a rotating plate, the rotating plate is rotatable relative to the at least first housing part, at least two pivotable filter holders are each arranged on the first housing part so as to be rotatable by means of a rotation axle, and each have a guide element oriented toward the rotating plate, wherein the rotating plate has, on its first plate surface, at least a first guide plane with a shaped, circumferential contact surface on a circumference so that, when the rotating plate is rotated, a pressure force of the shaped, circumferential contact surface on at least one of the two guide elements can pivot the associated filter holder into or out of the optical passage or position it in an initial position free from the optical passage. Furthermore, the invention relates to a camera head and to a retrofit kit for retrofitting a camera head and/or an endoscope.

### CROSS-REFERENCE TO RELATED APPLICATIONS

This Application claims the benefit Under 35 U.S.C. 119(a) to German Patent Application No. 10 2024 110 880.0, filed Apr. 18, 2024, the disclosure of which is incorporated herein by reference in its entirety.

### SUMMARY

The invention relates to a filter changing apparatus for an endoscopic camera, wherein the filter changing apparatus has at least a first housing part, a rotating plate with two opposing plate surfaces, at least a first rotating plate bearing, and at least two pivotable filter holders, each with a filter receptacle for an optical filter, wherein the first rotating plate bearing is arranged between the first housing part and a first plate surface of the rotating plate, the rotating plate is rotatable relative to the at least first housing part, and the at least first housing part, the at least first rotating plate bearing, and the rotating plate each have an optical passage along an optical axis, wherein the at least two pivotable filter holders are each arranged on the first housing part so as to be rotatable by means of a rotation axle, and each have a guide element oriented toward the rotating plate. Furthermore, the invention relates to a camera head for an endoscope and to a retrofit kit for retrofitting a camera head and/or an endoscope.

In medical and non-medical applications, observation instruments such as endoscopes are used to examine internal cavities of a human or animal body or of an industrial, technical item, such as a pipeline. For imaging, a camera head having an image sensor can be used together with the endoscope. In order to improve the image quality and/or make different observation modes possible, it is known to introduce different filters into the beam path of the observation instrument.

For example, in fluorescence imaging, the item to be examined is exposed to light with an excitation radiation, which excites a fluorophore, which has been previously applied to the item or is already present, to emit light of a certain emission wavelength, wherein the excitation wavelength and the emission wavelength are usually different. Normally, the emission wavelength is longer than the excitation wavelength. The emitted emission light is usually significantly weaker than other light sources, such as excitation fluorescence light or imaging white light. For these reasons, it is necessary to filter out unwanted wavelength bands by means of a filter so that, if possible, only the desired spectrum and/or the emission wavelength of the fluorophore reaches the camera head in fluorescence mode. For changing between different observation modes, two or more filters are usually introduced one after the other into the beam path of the observation optics, for which purpose various filter changers are known in principle.

DE 101 57 057 A1 discloses an apparatus for positioning at least one optical component within an endoscopic system with a housing, through which the optical axis of the endoscopic system runs and in which the at least one component is arranged, which can be pivoted into the beam path and out of it again about a pivot axis running substantially in parallel with a longitudinal axis of the housing, wherein the at least one component, for example a filter for a specific spectral wavelength range, is arranged on a carrier pivotable about the pivot axis. In this case, a smallest distance of an inner wall of the housing from the pivot axis is smaller than a greatest distance of the pivot axis to an outer edge of the at least one component. Due to the closer arrangement of the pivot axis of each pivotable carrier to the inner wall of the housing, the number of carriers for holding as many different filters as possible is limited by the spatially extensive pivoting movement within the limited space of the housing. In addition, the housing is firmly connected to a housing of the optical head of the endoscope. Another disadvantage is that, due to the spatially closer arrangement of the pivot axis to the inner wall of the housing, a large number of movable individual parts is required in order to introduce multiple filters into the beam path. This results in increased costs and installation effort. In addition, this increases the risk of wear, inaccuracies in the particular pivoting movement, and consequently malfunctions.

From the applicant's own prior art (German application number 10 2022 131 502.9), a filter changing apparatus for an endoscopic camera head with at least three optical filters is known, which filter changing apparatus has a groove with a non-circular guide track and a rotating element, wherein, by rotating the rotating element, carriers arranged one after the other in the groove are moved in each case for one filter or a pivoting movement of a carrier arm into and out of the beam path takes place by means of a guide element partially arranged in the groove, due to an at least partial movement along the non-circular guide track. The disadvantage here is that, for arranging multiple filters, a carrier arm with a guide element is necessary in each case so that, due to the larger number of guide elements or filter carriers arranged directly in the groove, disruptive influences on the function of the filter changing apparatus can occur due to the occurring frictional forces. The high frictional forces can cause jamming of the movement mechanism and/or wear with a negative effect on the pivoting-in and pivoting-out behavior as well as a locking position so that a malfunction or defect of the filter changing apparatus can occur. In addition, the pivotable carrier arms overlap so that a larger installation space is required along the optical axis.

The object of the invention is to improve upon the prior art.

The object is achieved by a filter changing apparatus for an endoscopic camera, wherein the filter changing apparatus has at least a first housing part, a rotating plate with two opposing plate surfaces, at least a first rotating plate bearing, and at least two pivotable filter holders, each with a filter receptacle for an optical filter, wherein the first rotating plate bearing is arranged between the first housing part and a first plate surface of the rotating plate, the rotating plate is rotatable relative to the at least first housing part, and the at least first housing part, the at least first rotating plate bearing, and the rotating plate each have an optical passage along an optical axis, wherein the at least two pivotable filter holders are each arranged on the first housing part so as to be rotatable by means of a rotation axle, and each have a guide element oriented toward the rotating plate, and the at least first rotating plate bearing has two recesses for the passage of one guide element each, and the rotating plate has, on its first plate surface, at least a first guide plane with a shaped, circumferential contact surface on a circumference so that, when the rotating plate is rotated, a pressure force of the shaped, circumferential contact surface on at least one of the two guide elements can pivot the associated filter holder into or out of the optical passage or position it in an initial position free from the optical passage.

This provides a filter changing apparatus by means of which at least two or more filters can be individually successively pivoted into and out of the beam path of an endoscopic camera quickly, precisely, and efficiently. This makes it possible to change between different filters quickly and nevertheless clearly. Above all, the simple rotation clockwise and/or counterclockwise makes possible a specified sequence of the filters and quick changing between different filters and thus different observation modes and imaging in quick succession.

It is particularly advantageous that, when the rotating plate is rotated and the pressure force of the shaped, circumferential contact surface acts on at least one guide element, a low frictional force occurs since the pressure force acts only partially on the outer surface of the guide element and in particular in sections and/or on one side of the outer surface of the guide element. Due to the small-area pressure force transmission, wear and, consequently, a malfunction or defect of the filter changing apparatus is prevented. Since each guide element moves along the shaped, circumferential contact surface of the corresponding guide plane during the rotation of the rotating plate, there is also a continuous change in the surface area on which the pressure force of the shaped, circumferential contact surface acts. Consequently, a continuous change of the pressure-loaded surface of the corresponding guide element takes place according to the pivoting-in and pivoting-out movement. This ensures a precise movement of the corresponding guide element along the shaped, circumferential contact surface of the guide plane and, consequently, a safe and precise pivoting-in and pivoting-out of the filter receptacle and/or of an optical filter of the corresponding pivotable filter holder into the optical passage.

Since only the material thicknesses of the rotating plate and of the corresponding rotating plate bearing are arranged along the optical axis, the filter changing apparatus has a small axial extent and thus a small size along the optical axis.

Furthermore, it is particularly advantageous that the design of the rotating plate with at least a first guide plane with a shaped, circumferential contact surface makes it possible for the filter holders to be pivoted in and out when rotating both clockwise and counterclockwise, and that the direction of rotation can be changed at any time. Since the shaped contact surface of the corresponding guide plane is circumferential and thus has no end, the rotating plate can be continuously rotated by a user and the corresponding guide element can be moved spatially and/or two-dimensionally without limitation by means of the pressure force of the shaped, circumferential contact surface, whereby a corresponding pivoting-in or pivoting-out movement or an initial or transitional position is imposed on the associated filter holder. The corresponding associated filter holder thus carries out a specific specified movement, remains in an initial position or assumes an initial position so that the position and movement of the at least two pivotable filter holders can be optimally coordinated in time and space. This makes it possible for at least two filter holders to be arranged in one plane and/or next to one another on a plate surface of the rotating plate.

The compact design of the rotating plate and of the at least one rotating plate bearing and the coordinated spatial arrangements and movements of the filter holders result in a very compact design of the filter changing apparatus. Above all, the filter changing apparatus has a small dimension along the optical axis due to the arrangement of the at least two filter holders in one plane.

It is particularly advantageous that the shape, geometry, and course of the corresponding guide plane with the shaped, circumferential contact surface, specifically the corresponding guide element and thus the associated filter holder, which can be rotated or pivoted about its rotation axle, specifies a pivoted-in, pivoted-out, transitional, initial, and/or rest position and/or movement in a defined manner. The geometry and the circumferential course of the specially shaped, circumferential contact surface of the corresponding guide plane defines a defined orientation and/or position of the corresponding filter holder relative to the optical axis and/or the optical passage and makes simultaneous movement of at least two filter holders on one of the two plate surfaces of the rotating plate possible. The course of the shaped, circumferential contact surface of the corresponding guide plane thus specifies defined, successive movements for the corresponding guide element in contact with this shaped, circumferential contact surface. The design and shape of the shaped, circumferential contact surface makes it possible for the filter holders to be individually pivoted in successively without the risk of the moving filter holders touching one another on a plate surface of the rotating plate. Consequently, the design of the shaped, circumferential contact surface of the corresponding guide plane thus specifies the spatial orientation of at least two filter holders arranged on a plate surface, and thus the temporal sequence of the movement of each filter holder and the changing of the filters.

In order to specifically convert the pressure force exerted by the shaped, circumferential contact surface of the corresponding guide plane onto the corresponding guide element into a desired pivoting movement or maintenance of an initial position, the at least two filter holders are arranged by means of their respective rotation axles at a spatially specified distance and in a spatial position relative to one another and/or to the shaped, circumferential contact surface.

An essential idea of the invention is based on arranging at least two filter holders which can be pivoted about their respective rotation axle on a distal-side or proximal-side housing part, wherein a respective guide element of each filter holder is directed inward and passes through an associated recess of a rotating plate bearing, which is arranged between the housing part and a rotating plate, wherein the rotating plate has, on its plate surface facing the guide elements, at least one guide plane with a shaped, circumferential contact surface so that, when the rotating plate is rotated, the shaped, circumferential contact surface exerts a pressure force on the corresponding guide element, whereby the guide element is moved spatially and a pivoting movement about the rotation axle or an initial position can be imposed on the associated filter holder. Due to the shape and the course of the shaped, circumferential contact surface, a spatial orientation and/or movement relative to the optical passage is specified for the corresponding filter holders by means of the pressure force and the associated guide element. In interaction with the pivotable arrangement of the at least two filter holders on the housing part by means of a respective rotation axle, a temporal progression and an order of changing the filters are imposed when the rotating plate is rotated. This provides a filter changing apparatus by means of which various filters, in particular multiple fluorescence filters and/or a white light filter, can be pivoted into and out of the beam path of an endoscopic camera precisely, quickly, efficiently, and with a long service life of the filter changing apparatus.

The following terminology is explained:

A “filter changing apparatus” is in particular an apparatus by means of which at least one of two or more filters can be moved into and out of the optical beam path. By means of the filter changing apparatus, two or more filters are pivoted into and/or out of the optical passage, in particular individually and successively. The filter changing apparatus can in particular be activated manually or automatically to change the filters by rotating the rotatable rotating plate. This means that a filter can thus be pivoted into and out of the optical passage automatically or manually by rotation. This makes it possible to change between at least two filter receptacles and/or filters of two filter holders, the guide elements of which are arranged on the contact surface of the same guide plane, two different guide planes of a plate surface, or a guide plane of the first plate surface and a guide plane of the second plate surface and/or are acted upon by means of the pressure force of the corresponding guide plane. The filter changing apparatus in particular has at least two optical filters, preferably at least three or four and, optionally, further optical filters. Optionally, the filter changing apparatus can also have a filter receptacle which does not have an optical filter and thus allows free passage through the optical beam path. The filter changing apparatus can thus also introduce an empty filter receptacle into the optical passage and into the beam path. Likewise, instead of omitting an optical filter, free passage can also be made possible by a non-filtering optical element, such as a glass pane. A glass pane used as a window can also have an anti-reflective coating. The filter changing apparatus can in particular be integrated in a camera or can be connectable as a separate apparatus, for example designed as a snap-on filter, to the camera and/or an endoscope. For automatically changing filters, the filter changing apparatus may have an operating element, such as a switch on its outer surface. As an alternative or in addition to the user visually detecting the filter change, the filter changing apparatus can also have a display element and/or a sensor, for example a Hall sensor.

An “optical filter” (also simply referred to as “filter”) is in particular an optical element which selects the incident radiation and/or incident beams on the basis of specific properties, such as a wavelength, a polarization state, an angle of incidence and/or a direction of incidence, and thus allows them to pass through or prevents them from passing through. Likewise, an optical filter can change the properties of the light passing through it, for example by converting circularly polarized light into linearly polarized light. In particular, an optical filter can block a specific spectral wavelength band. An optical filter may, for example, be a graduated filter, an edge filter, a polarizing filter or an interference filter. An interference filter in particular has a coating which blocks light of a certain spectral range or allows it to pass through. The optical filter can in particular be used as an observation filter and/or detection filter, fluorescence observation filter or excitation filter. The optical filter in particular comprises glass or a crystalline material. The optical filter may be planar or designed as a filter lens. In principle, instead of the optical filter, another optical element, such as a lens, an aperture, a polarizer or a similar optical element, can also be arranged in the filter changing apparatus and/or the filter receptacle.

The term “white light filter” is understood in particular to mean that a corresponding receptacle and/or position is free of an optical element or that an optical element in a corresponding receptacle and/or position is free of a filter function so that the light and/or white light is allowed to pass through, in particular unchanged. A white light filter allows white light to pass through, in particular without changing its light properties, in particular the wavelengths. A white light filter may also be a filter that blocks near-infrared light. A “white light filter” may also be a filter that filters the light in order to improve the quality of the image when illuminated with white light. For this purpose, a BG39 filter from the Schott company can be used, for example. By means of a white light filter, the light captured by an image sensor and/or a camera can thus also be adapted to a sensitivity curve and/or a specific sensitivity of the human eye.

A fluorescence observation filter (also referred to as a fluorescence filter) is in particular an optical polychroic interference filter for separating the emitted fluorescence light from the excitation light used. The fluorescence filter thus blocks the specific fluorescence excitation radiation and allows the fluorescence emission radiation to pass along the optical beam path. Preferably, the fluorescence filter completely blocks the excitation light while allowing the fluorescence emission light to pass through, which usually has a longer wavelength than the excitation light. A fluorescence filter is thus, in particular, an observation filter that filters out the excitation light that causes a fluorophore to glow. This is advantageous since the excitation light is general multiple orders of magnitude brighter than the resulting and/or emitted fluorescence light and would otherwise outshine it. A fluorescence filter may also be a blue filter, red filter, IR filter, or NIR filter.

The term “blue filter” is understood in particular to mean a filter that filters out the blue excitation light of a light source but at least predominantly allows the fluorescence light, in particular fluorescence light emitted by a fluorophore, to pass through. For example, when using the fluorophore FITC (fluorescein isothiocyanate, green derivative of fluorescein), excitation is carried out by means of an LED at a wavelength of 460 nm, wherein longer-wavelength fluorescence light with a maximum at approximately 520 nm in the green spectral range is emitted by the fluorophore. In order to be able to represent the emitted fluorescence light of the FITC well in the imaging, the blue excitation light is filtered out by means of the filter changing apparatus and/or the camera.

The term “red filter” is understood in particular to mean a filter that filters out the red excitation light of a light source but at least predominantly allows the fluorescence light, in particular fluorescence light emitted by a fluorophore, to pass through.

The term “IR filter” is understood in particular to mean a filter that filters out infrared excitation light of a light source but at least predominantly allows the fluorescence light, in particular fluorescence light emitted by a fluorophore, to pass through.

The term “NIR filter” is understood in particular to mean a filter that filters out near-infrared excitation light of a light source but at least predominantly allows the fluorescence light, in particular fluorescence light emitted by a fluorophore, to pass through.

An “optical passage” is in particular a hollow space in the filter changing apparatus through which light can pass. An optical passage is in particular a continuous opening through the rotatable rotating plate, the corresponding rotating plate bearing, further constituents, and/or the housing of the filter changing apparatus. The optical passage is in particular arranged around the center of the cross section of the rotatable rotating plate, the corresponding rotating plate bearing, and/or around the optical axis. The optical passage extends in particular along the optical axis. In particular, a filter receptacle and/or an optical filter can be arranged in front of and/or in the optical passage in the light propagation direction. Likewise, when light passes through, the optical passage may be free of an arranged optical filter and/or a receptacle. In principle, the optical passage can have any cross-sectional shape, but the optical passage is preferably circular in cross section.

An “optical axis” is in particular a line along which a degree of rotational symmetry exists in an optical system. The optical axis is in particular an imaginary line that defines a path along which light propagates through the filter changing apparatus and/or the camera toward an image sensor. Preferably, the optical axis runs through the curvature center of the particular pivoted-in filter and/or of a downstream lens system and/or objective system. However, the optical axis can also be bent and/or directed by a lens, an optical element and/or one of the optical filters. The optical beam path as a geometric course of light beams is in particular arranged in and/or around the optical axis and runs along, converges toward and/or disperses from the optical axis.

A “rotating plate” (also called “filter wheel”) is in particular a plate that is rotatable and has at least one guide plane on one plate surface or both plate surfaces. The rotating plate is in particular rotatable about its point of rotation and/or its axis of rotation and/or central axis. The axis of rotation of the rotating plate is preferably located in the optical axis. The rotating plate is in particular rotatable relative to the at least one rotating plate bearing and the at least one housing part. The rotating plate in particular has a bearing recess and/or an optical passage, which is or are arranged concentrically with the optical axis and/or the optical beam path. The rotating plate can in particular be driven and rotated by hand and/or by means of a drive unit and/or a motor. For this purpose, the outer circumferential surface may, for example, be driven as a drive surface by a drive unit and/or a gearing mechanism acting at and/or on this drive surface. Accordingly, the drive surface can be specifically designed for driving, for example can have external gears. The drive surface can also be specially designed for rotating by hand and, for example, not be circular but have multiple semicircular recesses on its outer circumference. For example, the rotating plate may have multiple, in particular six, protruding corners on its outer circumference between elongated, semicircular recesses. The rotating plate is in particular a substantially flat, planar component, with its opposing plate surfaces oriented substantially perpendicularly to the optical axis. The rotating plate in particular has a bearing recess and/or an optical passage through its material thickness at its center of the plate surfaces. The bearing recess serves to rotatably support the rotating plate. The bearing recess in particular has a larger diameter than a diameter of the optical passage of the rotating plate. In order to keep the size of the filter changing apparatus along the optical axis as small as possible, the rotating plate in particular has a low material thickness. The rotating plate has, in particular on at least one of its two plate surfaces, a guide plane which is flatly arranged on and/or connected to the plate surface. Accordingly, the rotating plate has a greater material thickness in the area of the guide plane(s) than on the rest of the plate surface. The rotating plate can also have a greater material thickness on its outer edge. Between the outer edge of the rotating plate and the center of the rotating plate, a rotating plate bearing can be arranged flush at the corresponding plate surface but without contact with the corresponding plate surface, in particular along the optical axis. The rotating plate and the at least one guide plane or the guide plane may be formed in multiple parts or in one piece. For example, the guide planes may be manufactured by a machining process, such as milling, from a plate-shaped workpiece for producing the rotating plate. The rotating plate with one or more guide planes may also be cast from a plastic material. One or more guide planes may also be firmly bonded, for example glued, to a raw plate for forming the rotating plate. In order to reduce the frictional forces between each contact surface of the guide plane of the rotating plate and the outer surface of the corresponding guide element, the rotating plate in particular comprises a material with a low coefficient of friction, for example aluminum or a polymeric material, such as PTFE. Due to the design of each guide plane with a shaped, circumferential and closed contact surface, the rotating plate has no end and can thus be rotated with any number of rotations in either of the two directions of rotation.

A “guide plane” is in particular a plate-shaped area and/or portion of the rotating plate or a plate-shaped component arranged on a plate surface of the rotating plate. In the area of the guide plane, the guide plane in particular increases the material thickness of the rotating plate along the optical axis. Each guide plane can substantially have a round or circular cross-sectional shape. On its outer circumference, the guide plane in particular has a contact surface. The contact surface is, in particular, an outer end face. One guide plane or two or more guide planes can in particular have an optical passage and/or be arranged around the optical passage of the filter changing apparatus on a corresponding plate surface. Alternatively or additionally, one or more guide planes can also be arranged all around with their contact surfaces, oriented inward toward the optical passage, in the outer area of the rotating plate. When arranged on the inside around the optical passage, the particular contact surface of the guide plane thus presses against the corresponding guide element from the inside and, when arranged in the outer area of the rotating plate with an inward directed contact surface, it accordingly presses against the corresponding guide element from the outside. When the rotating plate is rotated, the rotational movement of the rotating plate is transmitted by means of the contact surface and its resulting pressure force into a movement of the corresponding guide element according to the shape of the guide plane in a direction transverse to the optical axis and thus initiates a pivoting movement of the associated filter holder. Each guide plane can in particular have a substantially circular and/or annular basic shape. However, the guide plane can have different diameters and/or shaped portions along its outer circumference. Each guide plane can have one or more recesses in its contact surface and thus a smaller diameter in cross section in the area of each recess. Two or more guide planes of a plate surface may in particular have different diameters and/or material thicknesses. Each guide plane on both opposing plate surfaces of the rotating plate may in principle have the same design and/or shape. Preferably, however, identically designed guide planes on both plate surfaces of the rotating plate are arranged so as to be rotated relative to one another, and/or the filter holders are arranged on the corresponding housing parts so as to be rotated relative to one another, in order to make it possible for the filter receptacles of the filter holders to be alternately pivoted into the optical passage of the filter changing apparatus. Each guide plane, in particular with its contact surface, provides a curved path along which one or more guide elements of the filter holders move.

A “rotating plate bearing” is in particular an element for guiding the rotatable rotating plate within the housing of the filter changing apparatus. In particular, the rotating plate bearing and the rotating plate are free of an axially extended shaft. The rotating plate bearing is in particular a specially shaped plate-shaped component. The rotating plate bearing has, in particular centrally, an optical passage and/or a connecting part around the optical passage. In particular, a distal-side rotating plate bearing and a proximal-side rotating plate bearing can be connected to each other via a respective connecting part. The connecting part may, in particular, be a pipe section. The optical passage and/or a bearing recess of the rotating plate is in particular larger than the outer diameter of the connecting parts of the distal-side and proximal-side rotating plate bearings so that the two connecting parts of the proximal-side and distal-side rotating plate bearings can be arranged on the inside in the bearing recess and/or the optical passage of the rotating plate and the rotating plate can rotate around the outer surface of the connecting parts of the rotating plate bearings. The distal-side rotating plate bearing is in particular firmly connected to the distal-side housing part and the proximal-side rotating plate bearing is in particular firmly connected to the proximal-side housing part. Each rotating plate bearing is connected in particular in a form-fitting and/or force-fitting manner to the corresponding housing part. For example, each rotating plate bearing is screwed to the housing part. For each guide element, a rotating plate bearing in particular has a recess so that the guide element can contact the corresponding contact surface of the guide plane of the rotating plate. Each recess can be formed within the plate surface completely through the material thickness of the rotating plate bearing or through a cut in the outer edge of the rotating plate bearing. Each recess in particular has a curved and/or circumferential shape. When two opposing recesses are introduced into the outer edge of a circular plate, two plate-shaped wings of the rotating plate bearing accordingly remain.

In principle, it should be pointed out that the terms “first” and “second” filter holder, filter and other terms as well as “first” and “second” guide plane, contact surface, plate surface and other terms serve only to differentiate. For example, when moving a particular guide element when rotating the rotating plate, which filter receptacle of the associated filter holder is pivoted into the optical passage first depends on the direction of rotation clockwise or counterclockwise.

The term “initial position” (also called “rest position”) is in particular understood to mean an assumed or maintained position and/or orientation of a particular filter holder, free from the optical passage. In this case, the associated guide element is arranged in particular in a specified position of the shaped, circumferential guide groove, at which position no pressure force is exerted on this guide element and the associated filter holder thus does not carry out any rotational and/or pivoting movement about its rotation axle. In the initial position, the associated filter holder is thus arranged in its longitudinal direction with its outer side facing inward, in particular at a distance adjacent to the outer diameter of the optical passage. In the rest position, the particular filter holder is thus in particular oriented horizontally and located below or above the optical passage.

A “transitional position” is in particular a location on the contact surface of a guide plane and/or of multiple guide planes that are mounted on top of one another, at which location none of the filter holders is pivoted into the optical passage.

A “filter holder” is in particular an elongated element and/or an arm, which is arranged on the inner side of a housing part so as to be rotatable about its rotation axle. The filter holder may have a drop-shaped or club-shaped form, with the filter receptacle preferably being arranged at the widest end of the filter holder. The filter holder in particular has a first bore in one of its flat and/or planar sides, wherein the rotation axle is arranged and/or is connected in a firmly-bonded or force-fitting manner, for example pressed in, in this bore. This first bore may be partially or completely continuous through a material thickness of the filter holder transverse to its longitudinal direction. An opening of this first bore is in particular oriented toward the corresponding housing part. The rotation axle of each filter holder is arranged in particular at the end and/or on the opposite side to the filter receptacle of the filter holder along the longitudinal direction of the filter holder. The rotation axle may, for example, be designed as a shaft. Furthermore, on its flat side opposite the rotation axle, the filter holder in particular has a second bore, in which the guide element is accommodated and/or fastened. This side of the filter holder is in particular oriented toward the associated rotating plate bearing and the rotating plate. The filter holder has at least one filter receptacle for an optical filter. Preferably, each filter holder has exactly one filter receptacle at its end opposite the rotation axle. In particular, the filter holders have the same and/or different lengths in their longitudinal direction. The length of each filter holder depends in particular on the corresponding diameter of the associated guide plane, which contacts the guide element of the filter holder. In particular, the longitudinal direction of the filter holder is arranged substantially transversely to the optical axis.

A “filter receptacle” (also called a “receptacle”) is in particular a hollow body or cavity with a partial or complete external enclosure and/or border, into which hollow body or cavity an optical filter can be inserted and which enclosure and/or border at least partially encloses the optical filter at its circumference. A filter receptacle may, for example, be a short tubular body. The filter receptacle in particular forms a holder and/or a protective casing for the optical filter.

A “guide element” is in particular an element on which a pressure force of a shaped, circumferential contact surface of a guide plane can act. The guide element is in particular firmly or detachably connected to the associated filter holder. The guide element can in particular be connected directly to the filter holder or indirectly to the filter holder via a connecting element and/or a ball bearing. The longitudinal direction of the guide element is oriented in particular in parallel with the optical axis. In particular, the guide element has such a length that it extends in its longitudinal direction from the filter holder, through a recess of the corresponding rotating plate bearing to at least partially along the material thickness of the guide plane and/or the contact surface of the guide plane so that a pressure force of the contact surface can act on an outer surface of the guide element. In this case, the guide element does not necessarily have to extend in its longitudinal direction over an entire material thickness of the relevant guide plane and/or the contact surface of the guide plane. If two guide planes with different diameters are arranged on a plate surface, it is advantageous that the first guide element, which is assigned to the upper and/or distal or proximal guide plane, is designed to be at least slightly shorter than the length of the material thickness of this guide plane so that the end of the guide element does not hit the subsequent guide plane arranged below and/or more centrally with an at least partially larger diameter and cause increased friction. Accordingly, two or more guide elements, when in contact with the same guide plane of a plate surface or with a guide plane each with the same arrangement and material thickness, can have the same length in the longitudinal direction on both plate surfaces. Two or more guide elements can also have different lengths when in contact with guide planes arranged on top of one another and/or one after the other along the optical axis, in order to contact the intended contact surface of one of the guide planes. A guide element is in particular rod-shaped and/or cylindrical. A guide element may in particular be a guide pin or a guide bolt. Alternatively or additionally, the filter holders with their rotation axles can also be fastened to different levels of the housing part so that guide elements of the same length are guided to different distances to the contact surfaces. When the rotating plate is rotated, the associated guide element runs at least partially on the outside around the contour of the guide plane and thus along the shaped, circumferential contact surface of the guide plane.

A “housing part” is in particular a constituent of the filter changing apparatus, onto and/or to which the at least two pivotable filter holders are rotatably fastened. The housing part is in particular free of a guide plane. The housing part may be a distal or proximal housing part and/or a distal or proximal housing cover of the filter changing apparatus. The housing part may also be a base plate inside the housing. In particular, the rotating plate is arranged rotatably between the proximal and distal housing parts and/or covers in such a way that the outer surface of the rotating plate is freely accessible from the outside, at least on its outer circumference. The rotating plate may also have a larger diameter than one or both housing parts. In particular, the two proximal and distal housing parts are not connected directly to each other, but are connected inside the housing via the two rotating plate bearings. Each housing part has a bore on the inside, in particular for each rotation axle.

A “camera” (also called a “camera head”) is in particular a piece of equipment for receiving image light along an optical axis from an endoscope and for focusing the received image light on at least one image sensor. In addition to the at least one image sensor, the camera may in particular have an aperture or a window for letting through the received image light and a lens system for focusing the image light on the at least one image sensor. The image data recorded by at least one image sensor can in particular be transmitted electronically by the camera head to a display system and/or to an image processing unit in order to display the endoscopic image to the user. The camera may have means for recognizing the connected endoscope and for processing algorithms. A connector for connecting an endoscope to the camera can be arranged at the distal end of the endoscope and/or the proximal end of the camera head. The filter changing apparatus according to the invention itself may also be designed as a connector for connecting an endoscope to a camera.

An “endoscope” is in particular a medical or industrial piece of equipment for endoscopic examination and inspection of a human or animal body cavity and/or an industrial cavity, such as a tube. The endoscope in particular has a handpiece, a shaft, a light source, a light guide, a sensor and/or a camera. The endoscope is in particular a video endoscope, which has digital image recording and image transmission and thus an integrated or connectable camera. In addition to medical and veterinary applications, an endoscope and/or video endoscope may, however, also be used for industrial purposes, for example for visual inspection in hard-to-reach cavities. In industrial applications, an endoscope is often referred to as a borescope.

An “image sensor” is in particular a light-sensitive electronic component which is based on an internal photoelectric effect. By means of the image sensor, one or more images from the viewing area of the imaging apparatus are in particular recorded and converted into electronic signals. The image sensor has a sensor plane in the image plane of the optical system, of a lens system and/or of the objective. An electronic image sensor may in particular be a CCD sensor (charge-coupled device) or a CMOS sensor (complementary metal-oxide semiconductor).

The terms “distal-side” and “distal” are in particular understood to mean an arrangement and/or a corresponding end or portion that is remote from the user. Accordingly, when the filter changing apparatus is connected to an endoscope, the endoscope is arranged on the distal side. Accordingly, the terms “proximal-side” or “proximal” are understood to mean an arrangement or a corresponding end or portion near the user. When the filter changing apparatus is connected to a camera and/or a camera head, the camera and/or the camera head is accordingly arranged on the proximal side of the filter changing apparatus.

In a further embodiment, the filter changing apparatus has at least one resilient element assigned to each filter holder, wherein the at least one resilient element can act on the corresponding filter holder in such a way that a spring force of the at least one resilient element is directed against the pressure force of the shaped, circumferential contact surface.

This provides a filter changing apparatus with spring-loaded, in particular torsion-sprung, filter holders. By means of at least one resilient element or two or more resilient elements, it is ensured that the corresponding guide element of the filter holder does not lose contact with the contact surface of the guide plane. For this purpose, the resilient element can engage in the corresponding filter holder and an inner side of the housing part and thereby exert a spring force on the filter holder in order to press the guide element against the contact surface. Preferably, the spring force of each resilient element is substantially opposite to the pressure force of the shaped, circumferential contact surface. The term “substantially opposite” is understood to mean that the spring force and the pressure force do not have to be oriented toward each other from exactly opposite directions. Since the change in the circumferential shape of the contact surface along the contour of the contact surface causes the exerted pressure force to change in strength and/or direction in accordance with the change in shape, the opposing spring force does not always have to act in exactly the opposite direction, but it must be sufficient to ensure that the corresponding guide element is pressed against the contact surface.

Since each filter holder is spring-loaded with at least one resilient element, a second side wall opposite the shaped, circumferential contact surface and/or a guide groove is not required to guide the guide elements in a defined manner along the shaped, circumferential contact surface. Instead, the guide elements are pressed against the corresponding shaped, circumferential contact surface of the guide planes by applying a respective spring force by means of an associated resilient element. This minimizes the number of friction surfaces and halves them in comparison to a design with a guide groove. Consequently, the friction is minimized both on each shaped, circumferential contact surface of the guide plane and on the outer surface of the corresponding guide element. In addition to wear, the resilient element also prevents jamming of the corresponding guide element since the resilient element acts on the outside of the filter holder, but not on the guide element itself, and the guide element is thus only limited on one side by the contact surface.

Even if wear occurs on the shaped, circumferential contact surface and/or the corresponding guide element of the filter changing apparatus during long-term operation, the spring force of the resilient element ensures that, despite the resulting play, the corresponding guide element is optimally pressed against the shaped, circumferential contact surface so that the predefined pivoting-out or pivoting-in movement and/or pivoted-out or pivoted-in position as well as an initial and/or rest position of the corresponding filter holder are still maintained. Although a pivoting movement is made possible via the rotation axle of each filter holder, the precise maintenance of the desired movement and/or position of each filter holder is implemented through the interaction of the pressure force of the shaped, circumferential contact surface and the spring force of the at least one resilient element, thereby ensuring that the desired movement and/or position are precisely assumed and/or maintained. The at least one resilient element simultaneously makes it possible for the corresponding filter holder to snap into the pivoted-in position or the initial position. In particular, two resilient elements per filter holder make it possible for the corresponding filter holder to be held in the desired position and, in the pivoted-in state, concentrically with the beam path, and for a locking function to be implemented at the same time.

If the particular guide planes are arranged with the contact surfaces on the inside and/or around the optical passage and thus press the corresponding guide element outward, the at least one resilient element or the resilient elements press from the outside against the corresponding filter holder in the direction of the optical passage and/or beam path. In an alternative of the mechanism, the mode of action of the at least one resilient element or of the resilient elements can also be reversed. In this case, the particular guide plane is arranged on the outside of the circumference of the rotating plate and presses the filter holders inward in the direction of the optical passage and/or beam path via the guide elements. Accordingly, the at least one resilient element is or the resilient elements are arranged on the inside around the center and/or the optical beam path and, by means of the spring force, press the corresponding filter holder outward away from the optical passage and/or beam path.

A “resilient element” is in particular a technical component that is elastically deformable. The elastic deformation of the resilient element is in particular a bending and/or a torsion. The resilient element comprises, in particular, metal and/or plastic. The resilient element preferably comprises steel. The resilient element may, for example, be a leg spring. A leg spring is, in particular, a helically coiled wire spring with protruding straight ends and/or legs. One leg can act on a filter holder and the other leg can be fastened to the housing part. The legs are used in particular to introduce the torque that bends the wire. The helically coiled part of the leg spring can in particular be guided on an inserted cylinder and/or pin. A leg spring may also be a torsion spring. A torsion spring may be a straight, stretched torsion spring, such as a torsion bar spring, or a coiled torsion spring, such as a helical spring. The leg of the torsion spring that presses against the filter holder can in particular act in a groove along the outer contour of the filter holder. The number of helical turns of the leg spring and/or torsion spring can in particular be used to specify the spring force.

In order to further increase the number of filter holders and, in so doing, to avoid a restriction of movement and/or a collision of more than two pivotable filter holders in one plane on the first plate surface, the filter changing apparatus can have at least a second housing part, at least a second rotating plate bearing, and at least one further pivotable filter holder, in particular two further pivotable filter holders, arranged on the second housing part, wherein the second rotating plate bearing is arranged between the second housing part and a second plate surface of the rotating plate and the rotating plate has, on its second plate surface, at least a first guide plane with a shaped, circumferential contact surface on a circumference.

While maintaining a compact size of the filter changing apparatus, a filter change can thus be implemented between at least three and preferably four pivotable filter holders with corresponding filters.

Preferably, the filter holders and/or the guide planes on both sides of the rotating plate are arranged at an offset from one another and/or rotated relative to one another in the filter changing apparatus in order to make it possible for the filters to be pivoted in alternately by means of the filter holders.

Since the filter holders, their mounting, the rotating plate bearings, and the guide planes have a symmetric structure, the filter changing mechanism can be operated in both counterclockwise and clockwise rotation. In addition, for the user, this advantageously ensures that the movements of the filter holders are identical and only mirrored when rotating counterclockwise and clockwise.

In a further embodiment of the filter changing apparatus, the first plate surface and/or the second plate surface has or have a second guide plane with a shaped, circumferential contact surface so that a first guide element can be moved along the first contact surface of the first guide plane and a second guide element can be moved along the second contact surface of the second guide plane, of the first plate surface and/or of the second plate surface in each case.

This allows two filter holders to be moved separately and individually by means of their guide elements by means of two contact surfaces of two guide planes and thus to be pivoted in and/or out or held in an initial position. It is particularly advantageous that each guide plane can be designed specifically with regard to the different frequency of use of a filter holder and/or filter in the filter changing apparatus.

In order to adjust the pivoting movement and/or position of each filter holder and/or its associated filter individually, the first guide plane and the second guide plane of the first plate surface and/or of the second plate surface can have different diameters and/or differently shaped, circumferential contact surfaces.

In a further embodiment of the filter changing apparatus, the filter holders have the same length and/or a different length.

The filter holders of one plate surface can thus have the same length or a different length and, in each case, the same length as and/or a different length than the filter holders of the opposing plate surface.

In order to specify a defined position for pivoting each filter holder into the optical passage and a sequence of the related pivoting-in movement, the first guide plane and/or the second guide plane can each have at least one recess in a contact surface so that, when a guide element is arranged in the recess, the filter receptacle of the associated filter holder can be precisely pivoted into the optical passage of the rotating plate.

The shape and/or depth of the recess in the corresponding contact surface can thus be used to specifically specify the sequence of the pivoting-in movement and the temporal progression to reach the pivoted-in position depending on the rotational speed.

In order to pivot in a filter holder and thus a specific filter multiple times during one rotation of the rotating plate, two, three, four, or optionally further recesses can be introduced into a contact surface so that the corresponding filter is pivoted in multiple times regardless of the direction of rotation of the holder. Preferably, the recesses in a contact surface for pivoting in the same filter holder are designed in the same way.

A “recess” is in particular a recessed space and/or an opening partially or completely in the contact surface and/or the flat side or sides of a guide plane. A recess is in particular a cut into the contact surface and thus into the outer surface of a guide plane. In the area of the recess, the guide plane in particular has a smaller outer diameter in cross section. The associated guide element thus moves along the shaped recess of the contact surface so that, at a position in the recess at which the smallest diameter of the guide plane in cross section is present, the associated filter holder with its filter receptacle is pivoted into the optical passage. The recess is in particular specifically shaped and can, for example, be designed to taper conically toward the optical passage. Preferably, the recess has straight and/or curved portions. For example, the recess can be formed by multiple successive circular arc segments. In the case of two guide planes arranged next to one another on a plate surface, in addition to a more pronounced recess and thus a larger cut in the direction of the optical passage of the guide plane resting directly on the plate surface, the guide plane arranged above and/or on the distal or proximal side can also have, at the same position, a slightly smaller recess for the guide element of the guide plane arranged directly on the plate surface, in order to guide and/or position this guide element specifically when the pressure force is applied over a large proportion of its length. In addition, the guide plane arranged above and/or on the distal or proximal side has a more pronounced recess for the guide element assigned to this guide plane.

In a further embodiment of the filter changing apparatus, the first contact surface of the first guide plane, the second contact surface of the second guide plane, the at least one recess or the recesses are formed and/or arranged symmetrically.

Due to the symmetrical design, the temporal sequence and order of the successively pivoted-in filters is independent of the direction of rotation of the rotating plate.

In order to pivot in different filters at different frequencies during one rotation of the rotating plate, the first plate surface and the second plate surface can each have a second guide plane, each with a recess for pivoting in a filter holder with a first optical filter, and one of the two plate surfaces can have a first guide plane with four equally spaced recesses for pivoting in a filter holder with a second optical filter, so that one of the first optical filters and the second optical filter can in each case be pivoted in alternately one after the other during one rotation of the rotating plate.

In the event that the first optical filter is a fluorescence filter and the second optical filter is a white light filter, the white light filter, which is usually used most frequently, can be pivoted in alternately with a fluorescence filter of the corresponding filter holder assigned to the second guide plane of the first plate surface and of the second plate surface. By pivoting in the white light filter by means of the associated filter holder between each pivoted-in position of a corresponding fluorescence filter, the user can quickly change between a desired fluorescence mode and the white light mode. By interposing the white light filter, the user is clearly shown a change between similar or different fluorescence filters. This reduces the risk of inadvertently working in an incorrect observation mode.

In this regard, it should be pointed out that the first optical filter and the second optical filter can in principle be any type of filter and that the number of recesses in each contact surface of a particular guide plane can be specifically selected and designed differently than the number of recesses of another guide plane in order to specifically pivot in one or more filter holders more than once per rotation of the rotating plate.

Since two filter holders assigned to a plate surface have different lengths, only one guide element engages with a contact surface of the first guide plane or of the second guide plane at a time, and two filter holders can thus be moved independently of one another on the one plate surface.

In a further embodiment of the filter changing apparatus, the first guide plane and/or the second guide plane of the first plate surface and/or of the second plate surface are designed such that, in a transitional position, the optical passage is free of a pivoted-in filter holder.

Each guide plane thus has a position at which no filter is in the beam path. This transitional position is achieved in particular if a second guide element is arranged exactly opposite a recess on the contact surface of the same guide plane, wherein there is no recess at this transitional position and/or there is a maximum diameter of this guide plane and the first guide plane is also free of a recess at this position.

In order to connect both halves of the pivoting mechanism and thus the proximal housing part and the distal housing part, the first rotating plate bearing can have a first connecting element and the second rotating plate bearing can have a second connecting element, wherein the first connecting element and the second connecting element can be connected to each other in a form-fitting and/or force-fitting manner.

The term “form-fitting” is in particular understood to mean that the two connecting elements engage in each other to form the connection. In a form-fitting connection, one of the two connecting elements in particular blocks the movement of the other connecting element. This means that there is a blocking in at least one direction. The form-fitting connection may, for example, be a tongue-and-groove connection, a key, a toothing, or a rotation blocking.

The term “force-fitting” is understood in particular to mean a connection in which a normal force acts on the connecting surfaces of the two connecting elements and their relative displacement is prevented as long as the counterforce caused by static friction is not exceeded. A force-fitting connection may in particular be a screw connection or a clamped connection.

The connection point of the two connecting elements can form the bearing point for the rotating plate. For example, if the connecting elements are designed as pipe sections, the rotating plate with its central bearing recess and/or its optical passage can rotate on the outside around the outer surface of the tubular connection point.

In a further embodiment of the filter changing apparatus, the first housing part and the second housing part are connected to each other in their interior so that the rotating plate can be freely contacted and/or rotated over its entire circumference from the outside.

This gives the user full access to the rotating plate over the entire outer circumference, and there are no fastening elements that hinder the gripping and/or contacting of the outer circumference of the rotating plate. Consequently, the rotating plate can be optimally driven manually and/or by motor from the outside. The rotating plate is thus freely accessible and easy to handle.

In order to operate the filter changing apparatus without contact, the first housing part and the second housing part can form a hermetically sealed housing, wherein the rotating plate is arranged within the housing and has at least one counter-magnetic element so that rotation of the rotating plate can be caused by means of an assigned magnetic device outside the housing.

This makes the filter changing apparatus easier to clean and autoclave.

In a further aspect of the invention, the object is achieved by a camera head for an endoscope, wherein the camera head has an image sensor, an opening for receiving light of an image along an optical path, and an optical lens system for focusing the light on the image sensor, wherein the camera head has at least one filter changing apparatus described above.

This provides a camera head on or in which a compact, space-saving filter changing apparatus is arranged. If at least one filter changing apparatus is arranged directly in the camera head, the camera head can be detachably connected to different types of endoscopes. For example, the filter changing apparatus can be integrated into the camera head by installing it instead of a bayonet closure. This can minimize an extension of the beam path. Of course, the camera head can also have two or more filter changing apparatuses in series in one optical path and/or along the optical axis if the simultaneous use of two or more filters in the beam path is desired, in particular in multispectral imaging and/or in a broad application of different fluorophores in fluorescence imaging.

In order to adjust a currently used filter configuration or adapt it to desired different observation modes, the camera head may comprise a detection unit for detecting an identification of each optical filter in the optical path. Likewise, the camera head can have a control unit for adjusting the rotational speed of the rotating plate and for checking and/or adjusting the particular optical filter, arranged in the beam path, according to the operating mode selected at the time.

In a further aspect of the invention, the object is achieved by a retrofit kit for retrofitting a camera head and/or an endoscope, wherein the retrofit kit has at least one filter changing apparatus described above, so that the filter changing apparatus can be arranged between a proximal end of the endoscope and a distal end of the camera head.

This provides a retrofit kit (also referred to as an “adapter”) with at least one filter changing apparatus, which retrofit kit simultaneously serves both as a connector between an existing endoscope and an existing camera head and to make different observation modes possible. In addition, the retrofit kit may also comprise two or more filter changing apparatuses arranged in series between the endoscope and the camera head, or one filter changing apparatus may be replaced by another filter changing apparatus to make different applications and/or observation options possible.

### BRIEF DESCRIPTION OF THE DRAWINGS

The invention is explained in more detail below with reference to exemplary embodiments. In the figures:

FIG. 1 shows a schematic three-dimensional detail view of an endoscope system with an endoscope, a filter changer and a camera head,

FIG. 2 shows a three-dimensional representation of a filter changer in side view with an endoscope receptacle and a camera receptacle,

FIG. 3 shows an exploded view of constituents of the filter changing apparatus,

FIG. 4 shows a highly schematic plan view of a proximal side of a filter wheel,

FIG. 5 shows a highly schematic plan view of a distal side of the filter wheel,

FIG. 6 shows a highly schematic, three-dimensional representation of the proximal side of the filter wheel,

FIG. 7 shows a highly schematic representation of a proximal rotating disk bearing,

FIG. 8 shows a highly schematic representation of a distal rotating disk bearing, and

FIG. 9 shows a highly schematic representation of an alternative of a proximal rotating disk bearing with recesses on the inside.

### DETAILED DESCRIPTION

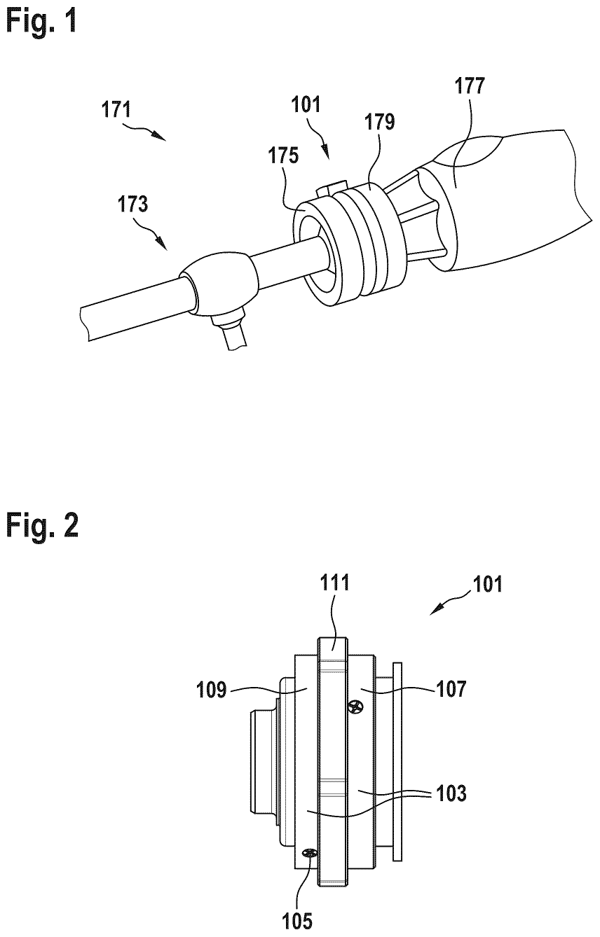

An endoscope system 171 has a camera head 177, a filter changer 101 and an endoscope 173. The filter changer 101 is connected to the camera head 177 by means of a camera receptacle 179 and to the endoscope 173 by means of an endoscope receptacle 175 (FIGS. 1 and 2).

The filter changer 101 has a housing 103 with a distal cover half 107 and a proximal cover half 109, between which a filter wheel 111 is arranged. By means of screws 105 directed inward, the distal cover half 107 is connected to a distal rotating disk bearing 130 and the proximal cover half 109 is connected to a proximal rotating disk bearing 133 inside the housing 103. As a result, the filter wheel 111 between the distal cover half 107 and the proximal cover half 109 can be freely contacted and rotated from the outside.

The distal cover half 107 and the proximal cover half 109 each have an optical passage 127 concentric with an optical axis 129. The proximal rotating disk bearing 133 is arranged between an inner side of the proximal cover half 109 and a proximal wheel surface 115 of the filter wheel 111, and the distal rotating disk bearing 130 is arranged between a distal wheel surface 113 and an inner surface of the distal cover half 107. A first filter holder 141 and a second, long filter holder 142 are fastened to the inner side of the proximal cover half 109, and a third filter holder 143 and a fourth filter holder 144 are fastened to the inner side of the distal cover half 107. The filter holders 141, 142, 143, and 144 are each drop-shaped and have a bore 145 at their narrower end. In each bore 145, a rotation axle 149 is accommodated, which is fastened to the inner side of the distal cover half 107 or the proximal cover half 109. Opposite the respective bore 145, the first filter holder 141 has a first filter receptacle 151, the second filter holder 142 has a second filter receptacle 152, the third filter holder 143 has a third filter receptacle 153, and the fourth filter holder 144 has a fourth filter receptacle 154 for one optical filter each. Between the bore 145 and the corresponding filter holder 151, 152, 153, 154, each filter holder 141, 142, 143, 144 has a long guide bolt 155 or a short guide bolt 157.

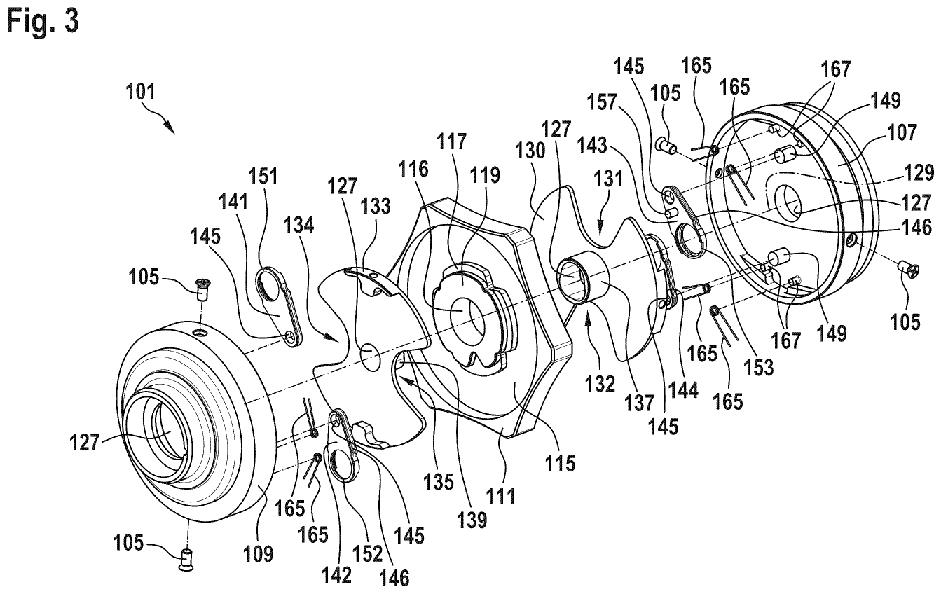

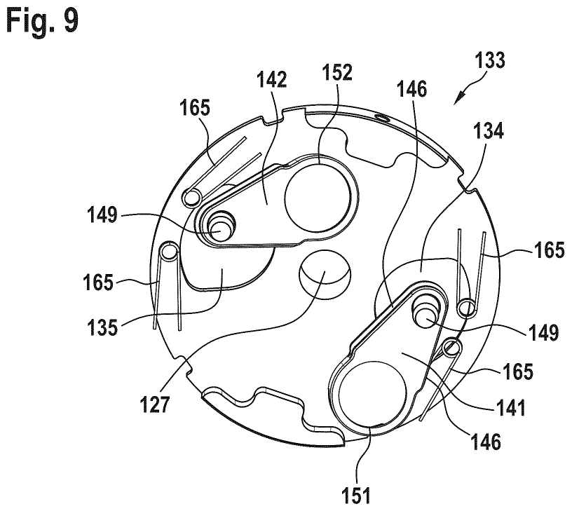

Two torsion springs 165 are arranged on both sides of each rotation axle 149, wherein one leg of each torsion spring 165 is connected on the inside to the inner side of the corresponding distal cover half 107 or proximal cover half 109 and the other leg engages in a groove 146, which is introduced on both sides circumferentially around the conical portion and thus around the rotation axle 149 of each filter holder 141, 142, 143, 144 into its respective outer surface. Each torsion spring 165 is fastened with its terminal winding to the inner surface of the distal cover half 107 or the proximal cover half 109 by means of a retaining pin 167 (FIG. 3 and FIG. 9).

The distal-side rotating disk bearing 130 has a first recess 131 and a second recess 132, which are arranged opposite each other so that the distal rotating disk bearing 130 has a shape with two wings. An optical passage 127 is arranged in the center of the distal rotating disk bearing 130, and a first connecting element 137 is arranged around the optical passage 127 (FIG. 8). The proximal rotating disk bearing 133 is similar in its basic shape with a third recess 134 and an opposing fourth recess 135 (FIG. 7). In its center, the proximal rotating disk bearing 133 also has an optical passage 127 and a second connecting element 139 arranged around the optical passage 127. The first connecting element 137 of the distal rotating disk bearing 130 and the second connecting element 139 of the proximal rotating disk bearing 133 are designed to be complementary to each other, wherein, in the assembled state, the second connecting element 139 is accommodated in the first connecting element 137. As a result, a form-fitting connection is formed between the first connecting element 137 and the second connecting element 139. In the assembled state, an outer surface of the first connecting element 137 is surrounded by a bearing recess 116 of the filter wheel 111.

In an alternative shown in FIG. 9 of a proximal rotating disk bearing 133, the third recess 134 and the fourth recess 135 are not arranged on the outer edge of the proximal rotating disk bearing 133 as shown in FIG. 7, but are formed to be circumferential further inward and to penetrate through a plate surface of the alternative of the proximal rotating disk bearing 133. A distal rotating disk bearing 130 is designed equivalently in this alternative.

The first recess 131 and the second recess 132 of the distal rotating disk bearing 130 as well as the third recess 134 and the fourth recess 135 of the proximal rotating disk bearing 133 each make it possible for a long guide bolt 155 and/or a short guide bolt 157 of the filter holders 141 to 144 to pass through to the corresponding distal wheel surface 113 or proximal wheel surface 115 of the filter wheel 111.

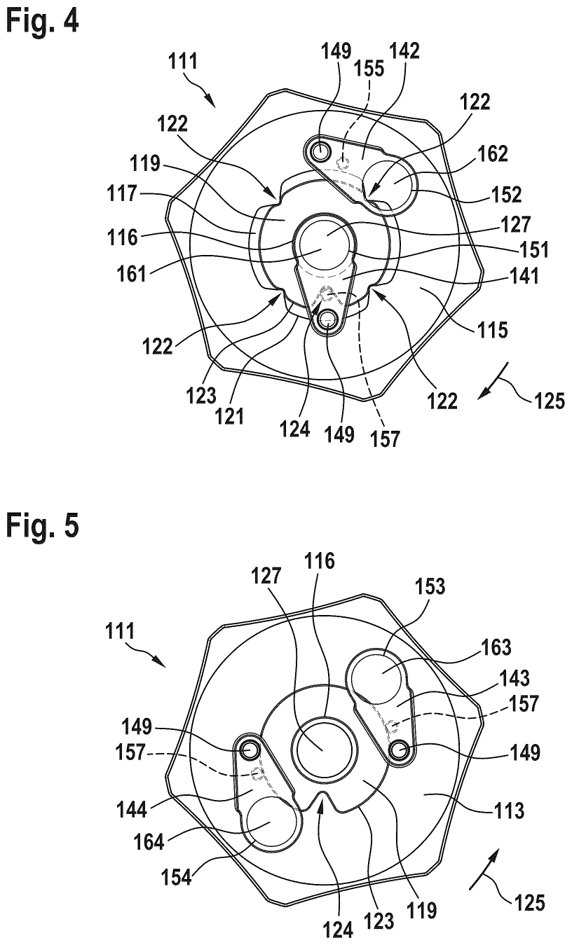

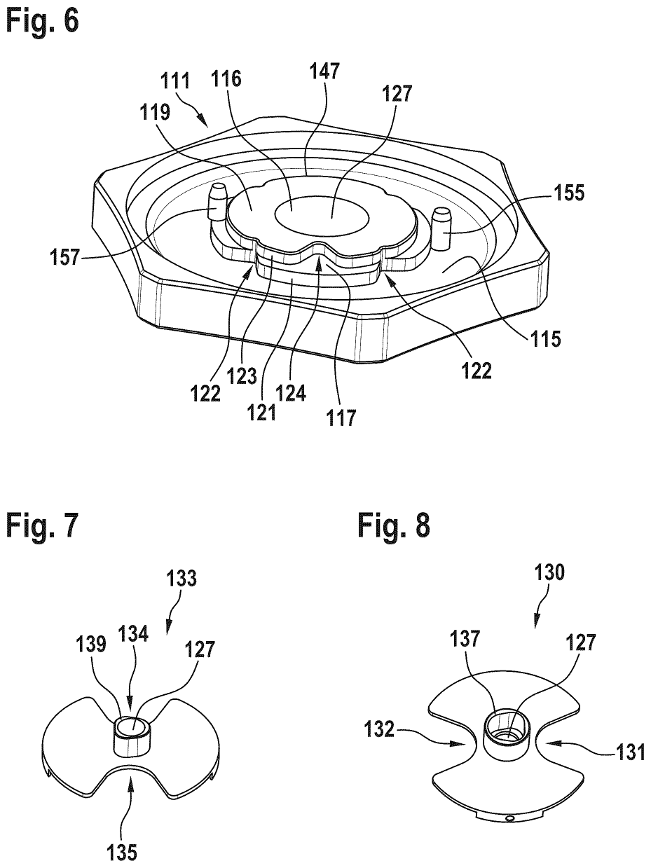

The filter wheel 111 has, on its proximal wheel surface 115, on the inside, the bearing recess 116 with a larger diameter than the optical passage 127 of the filter changer 101. A first guide plane 117 is arranged around the bearing recess 116 directly on the proximal wheel surface 115. On its circumference, the first guide plane 117 has a first contact surface 121, into which four first cuts 122 are introduced, evenly distributed over the circumference of the first guide plane 117. A second guide plane 119 with a deep second cut 124 in its second contact surface 123 is arranged on the first guide plane 117. Opposite this second cut 124, a transitional position 147 is arranged, at which both contact surfaces are free of a cut. When the short guide bolt 157 rests on this transitional position 147, all filter holders 141, 142, 143, 144 are pivoted out of the optical passage 127. Furthermore, smaller recesses (not further identified in FIG. 6) corresponding to the first cuts 122 of the first guide plane 117 are arranged in the second contact surface 123. A short guide bolt 157 rests with its outer surface laterally on the second contact surface 123 of the second guide plane 119. A long guide bolt 155 rests on the first contact surface 121 of the first guide plane 121. The short guide bolt 157 is connected to the first filter holder 141, in whose first filter receptacle 141 a first filter 161 for a fluorescence mode is accommodated. The long guide bolt 155 is connected to the long second filter holder 142, in whose second filter receptacle 152 a second filter 162 for a white light mode is accommodated (FIG. 4).

On its distal wheel surface 113, the filter wheel 111 has a second guide plane 119, as the only guide plane arranged directly on the distal wheel surface 113, around the bearing recess 116 with the internal optical passage 127. The second guide plane 119 of the distal wheel surface 113 is designed similarly to the second guide plane of the proximal wheel surface 115 but has only a second cut 124 in its second contact surface 123. Except for this second cut 124, the contact surface 123 has a circular circumference. The short guide bolts 157 of the third filter holder 143 and of the fourth filter holder 144 are arranged on this contact surface 123. The third filter holder 143 has, in its third filter receptacle 153, a third filter 163 for a different fluorescence mode, and the fourth filter holder 144 has, in its fourth filter receptacle 154, a fourth filter 164 for a further fluorescence mode.

The following operations are carried out by means of the filter changer 101 and the endoscope system 171.

When the filter wheel 111 is rotated by a user by contacting the outer surface of the filter wheel 111 outside the housing 103 in a clockwise rotation direction 125, the long guide bolt 155 of the long second filter holder 142 is moved along the first contact surface 121 and the short guide bolt 157 of the first filter holder 141 is moved along the second contact surface 123 on the side of the proximal wheel surface 115 (FIG. 4). Accordingly, on the side of the distal wheel surface 113, the two short guide bolts 157 of the third filter holder 143 and of the fourth filter holder 144 both move along the second contact surface 123 of the second guide plane 119 (FIG. 5). Each short guide bolt 157 and the long guide bolt 155 are each pressed against the corresponding contact surface 121, 123 by means of the torsion springs 165 assigned to the corresponding filter holder 141 to 144, in that the corresponding free leg of the corresponding torsion spring 165 presses in the corresponding groove 164 against the associated filter holder 141 to 144.

In the state shown in FIG. 4, the short bolt 157 of the first filter holder 141 lies in the second cut 124 of the second guide plane 119 on the proximal wheel surface 115 so that the first filter receptacle 151 with the first filter 161 is pivoted into the optical passage 127 and a fluorescence mode is accordingly used by the user. Meanwhile, the second filter holder 142 is pivoted out. Likewise, the third filter holder 143 and the fourth filter holder 144 on the opposite distal wheel surface 113 are pivoted out and are in an initial position.

Starting from the positions of the four filter holders 141, 142, 143, 144 shown in FIGS. 4 and 5, when the filter wheel 111 is rotated in the rotation direction 125, on the proximal wheel surface 115, the long guide bolt 155 is rotated along the first contact surface 121 in the rotation direction 125 up to the subsequent next first cut 122, wherein the short bolt 157 simultaneously moves along the second contact surface 123 in the rotation direction 125 and is pivoted out by leaving the second cut 124. The third filter holder 143 and the fourth filter holder 144 on the distal wheel surface 113 are further rotated when rotating in the rotation direction 125, which is counterclockwise due to the opposite arrangement, but they remain, free from the second cut 124, on the outer circumference of the second guide plane 119. When the first cut 122 is reached in the clockwise rotation direction 125, the long guide bolt 155 thus pivots into the first cut 122 and, consequently, the second filter receptacle 152 with the second filter 162 for a white light mode is pivoted into the optical passage 127. Upon further rotation in the rotation direction 125, the fourth filter receptacle 154 of the fourth filter holder 144 on the distal plate surface 133 is subsequently pivoted into the optical passage 127 when the short guide bolt 157 reaches the second cut 124. Upon further rotation in the rotation direction 125, the second long filter holder 142 with the second filter 162 for the white light mode is snapped in again on the proximal wheel surface 115 due to the four evenly spaced first cuts 122 in the first contact surface 121 of the first guide plane 117. Thus, upon further rotation in the rotation direction 125, after the first filter 161, the third filter 163, or the fourth filter 164 has been pivoted in for a fluorescence mode, the second filter holder 142 with a second filter 162 for a white light mode is always pivoted in. This clearly shows the user a change between the different fluorescence modes and the white light mode.

This provides a filter changer 101 by means of which changing between different filters 161, 162, 163, 164 can take place quickly, efficiently, and precisely one after the other and which can be used with different types of endoscopes 173 and camera heads 177.

### LIST OF REFERENCE SIGNS

101 filter changer
103 housing
105 screw
107 distal cover half
109 proximal cover half
111 filter wheel
113 distal wheel surface
115 proximal wheel surface
116 bearing recess
117 first guide plane
119 second guide plane
121 first contact surface
122 first cut
123 second contact surface
124 second cut
125 rotation direction
127 optical passage
129 optical axis
130 distal rotating disk bearing
131 first recess
132 second recess
133 proximal rotating disk bearing
134 third recess
135 fourth recess
137 first connecting element
139 second connecting element
141 first filter holder
142 second filter holder (long)
143 third filter holder
144 fourth filter holder
145 bore
146 groove
147 transitional position
149 rotation axle
151 first filter receptacle
152 second filter receptacle
153 third filter receptacle
154 fourth filter receptacle
155 long guide bolt
157 short guide bolt
161 first filter (fluorescence)
162 second filter (white light)
163 third filter (fluorescence)
164 fourth filter (fluorescence)
165 torsion spring
167 retaining pin
171 endoscope system
173 endoscope
175 endoscope receptacle
177 camera head
179 camera receptacle

1. A filter changing apparatus for an endoscopic camera, wherein the filter changing apparatus comprises:
at least a first housing part,
a rotating plate with two opposing plate surfaces,
at least a first rotating plate bearing, and
at least two pivotable filter, each with a filter receptacle, for an optical filter, wherein the first rotating plate bearing is arranged between the first housing part and a first plate surface of the rotating plate, the rotating plate is rotatable relative to the at least first housing part, and the at least first housing part, the at least first rotating plate bearing, and the rotating plate each have an optical passage along an optical axis, wherein the at least two pivotable filter holders are each arranged on the first housing part so as to be rotatable by means of a rotation axle, and each have a guide element oriented toward the rotating plate, wherein the at least first rotating plate bearing has two recesses for the passage of one guide element each, and the rotating plate has, on its first plate surface, at least a first guide plane with a shaped, circumferential contact surface on a circumference so that, when the rotating plate is rotated, a pressure force of the shaped, circumferential contact surface on at least one of the two guide elements can pivot the associated filter holder into or out of the optical passage or position it in an initial position free from the optical passage.

2. The filter changing apparatus according to claim 1, wherein the filter changing apparatus has at least one resilient element assigned to each filter holder, wherein the at least one resilient element can act on the corresponding filter holder in such a way that a spring force of the at least one resilient element is directed against the pressure force of the shaped, circumferential contact surface.

3. The filter changing apparatus according to claim 1, wherein the filter changing apparatus has at least a second housing, at least a second rotating plate bearing, and at least one further pivotable filter holder, in particular two further pivotable filter holders, arranged on the second housing part, wherein the second rotating plate bearing is arranged between the second housing part and a second plate surface of the rotating plate, and the rotating plate has, on its second plate surface, at least a first guide plane with a shaped, circumferential contact surface on a circumference.

4. The filter changing apparatus according to claim 1, wherein the first plate surface and/or the second plate surface has or have a second guide plane with a shaped, circumferential contact surface so that a first guide element can be moved along the first contact surface of the first guide plane and a second guide element can be moved along the second contact surface of the second guide plane, of the first plate surface and/or of the second plate surface in each case.

5. The filter changing apparatus according to claim 4, wherein the first guide plane and the second guide plane of the first plate surface and/or of the second plate surface have different diameters and/or differently shaped, circumferential contact surfaces.

6. The filter changing apparatus according to claim 1, wherein the filter holders have the same length and/or a different length.

7. The filter changing apparatus according to claim 1, wherein the first guide plane and/or the second guide plane each have at least one recess in their contact surface so that, when a guide element is arranged in the recess, the filter receptacle of the associated filter holder can be precisely pivoted into the optical passage of the rotating plate.

8. The filter changing apparatus according to claim 1, wherein the first contact surface of the first guide plane, the second contact surface of the second guide plane, the at least one recess or the recesses are formed and/or arranged symmetrically.

9. The filter changing apparatus according to claim 1, wherein the first plate surface and the second plate surface each have a second guide plane, each with a recess for pivoting in a filter holder with a first optical filter, and one of the two plate surfaces has a first guide plane with four equally spaced recesses for pivoting in a filter holder with a second optical filter, so that one of the first optical filters and the second optical filter can in each case be pivoted in alternately one after the other during one rotation of the rotating plate.

10. The filter changing apparatus according to claim 1, wherein the first guide plane and/or the second guide plane of the first plate surface and/or of the second plate surface are configured such that, in a transitional position, the optical passage is free of a pivoted-in filter holder.

11. The filter changing apparatus according to claim 1, wherein the first rotating plate bearing has a first connecting element and the second rotating plate bearing has a second connecting element, wherein the first connecting element and the second connecting element can be connected to each other in a form-fitting and/or force-fitting manner.

12. The filter changing apparatus according to claim 1, wherein the first housing part and the second housing part are connected to each other in their interior so that the rotating plate can be freely contacted and/or rotated over its entire circumference from the outside.

13. The filter changing apparatus according to claim 1, wherein the first housing part and the second housing part form a hermetically sealed housing, wherein the rotating plate is arranged within the housing and has at least one counter-magnetic element so that rotation of the rotating plate can be caused by means of an assigned magnetic device outside the housing.

14. A camera head for an endoscope, wherein the camera head has an image sensor, an opening for receiving light of an image along an optical path, and an optical lens system for focusing the light on the image sensor, wherein the camera head has at least one filter changing apparatus according to claim 1.

15. A retrofit kit for retrofitting a camera head and/or an endoscope, wherein the retrofit kit has at least one filter changing apparatus according to claim 1 so that the filter changing apparatus can be arranged between a proximal end of the endoscope and a distal end of the camera head.
